(19) United States
(12) Patent Application Publication
(10) Pub. No.: US 2025/0235509 A1
(43) Pub. Date: Jul. 24, 2025

(54) Formulation and Method of Preparing Glucagon-like Peptide-1 (GLP-1) for Oral Administration

(71) Applicant: Men Hwei Tsai, Alhambra, CA (US)

(72) Inventor: Men Hwei Tsai, Alhambra, CA (US)

(21) Appl. No.: 18/420,098

(22) Filed: Jan. 23, 2024

(51) Int. Cl. A61K 38/26; A61K 9/00; A61K 47/52; A61P 3/04; A61P 3/10

(52) U.S. Cl. CPC A61K 38/26; A61K 9/0053; A61K 47/52; A61P 3/04; A61P 3/10

(57) ABSTRACT

A zinc-glucagon-like peptide composition for inducing an insulinotropic response in a patient through oral administration is disclosed. The composition includes a glucagon-like peptide (GLP-1) such as a GLP-1 analogue, as a polypeptidic chain of amino acids joined by peptide linkages and ionically bound to zinc. The glucagon-like peptide is first incubated with a chelating agent and thereafter incubated with a zinc salt solution, wherein the zinc is ionically bound to the chelated glucagon-like peptide. The zinc-glucagon-like peptide is then packaged for oral administration and orally administered for intestinal absorption of the glucagon-like peptide.

### CROSS-REFERENCE TO RELATED APPLICATIONS

NONE

### FIELD OF THE INVENTION

The present invention relates generally to a formulation for glucagon-like peptide-1 (GLP-1) that is available to be administered orally, as well as a method of turning glucagon-like peptide-1 (GLP-1) into a form that is available for oral administration.

### BACKGROUND OF THE INVENTION

Proper glucose metabolism is a prerequisite for human health. Prolonged periods of hypo-glycemia or hyperglycemia result in a variety of known diseases including diabetes, vascular damage, and other conditions. Insulin is the primary hypoglycemic hormone for reducing blood glucose. Produced by pancreatic islet beta cells, insulin promotes the absorption of blood glucose by various tissues. Insulin is one of the most well-known macromolecules, primarily used by the liver, fat, and muscles to regulate the concentration of blood glucose. The secretion of insulin regulates the concentration of glucose in the blood, such that the level of blood glucose is always stable whether the body is undergoing fasting or feeding.

Glucagon is the primary hyperglycemic hormone for increasing blood glucose. Produced in the pancreas by alpha cells, glucagon stimulates, for example, glycogenolysis in the liver, converting stored glycogen into glucose, among other functions. Properly metabolized glucose levels depend largely on the interplay between these two hormones.

Incretins, a group of metabolic hormones, stimulate the pancreas to secrete insulin, inhibiting glucose release by other organs. One such incretin, glucagon-like peptide 1 (GLP-1), has been found to enhance insulin secretion as well as activating satiety receptors in the brain. GLP-1 is a peptide hormone cleaved from proglucagon, which is secreted in the pancreas. GLP-1 has multiple actions on glucose, mediated by GLP-1 receptors, and the major source of natural GLP-1 is from cells found in the lining of the small intestine. GLP-1 acts by encouraging insulin release in the pancreas, increasing the volume of insulin producing cells (beta cells) in the pancreas, and reducing the release of glucagon (a hormone that increases blood glucose). Due to its action on the brain, GLP-1 also increases satiety (the feeling of fullness) during and between meals by acting on appetite centers in the brain and by slowing the emptying of the stomach. As a result, GLP-1 has the effects of lowing blood glucose and reducing body weight. Hence, GLP-1 and its analogs may be used as part of a treatment regimen for diabetes and obesity, among other diseases.

Native GLP-1 has a very short plasma half-life. Analogues of human GLP-1 have therefore been developed to lengthen that half-life. Semaglutide, Liraglutide, and Tirzepatide are the three most recently approved GLP-1 analogs. The current method for administering one of these GLP-1 analogs to a patient is through subcutaneous injection, where the peptide is injected into the tissue between the skin and muscle with a small needle. This injection is generally done in a patient's abdomen to enable an effective amount of the GLP-1 analog to be absorbed by the body, and is currently the most common and preferred method of effectively administering GLP-1 analogs to patients.

Due to the inability of GLP-1 analogs to survive the acidic conditions of the stomach, they cannot be administered orally. Oral administration, however, would be preferred over subcutaneous injection due to the less intrusive nature of oral administration versus injections. As such, there is a need for a GLP-1 analog composition that may be administered orally and still effectively be absorbed by the body. Additionally, there is a need for a technique to make a GLP-1 analog capable of being orally administered and effectively absorbed by the body. Additionally, there is a need for a technique to convert subcutaneous injection GLP-1 analog products on the market, such as Semaglutide, Liraglutide, and Tirzepatide into a GLP-1 analog that may be effectively administered orally.

### SUMMARY OF THE INVENTION

A zinc-glucagon-like peptide composition for inducing an insulinotropic response in a patient through oral administration is disclosed. The composition comprises a glucagon-like peptide, which may be a glucagon-like peptide analogue, comprising a polypeptidic chain of amino acids joined by peptide linkages. The glucagon-like peptide is ionically bound to the zinc. The glucagon-like peptide is first incubated with a chelating agent. Thereafter, the glucagon-like peptide is further incubated with a zinc acetate solution, or any other solution containing zinc salts, such as zinc chloride, zinc sulfate, etc. The second incubation results in the zinc being ionically bound to the chelated glucagon-like peptide, thereby creating the zinc-glucagon-like peptide which is capable of oral administration and intestinal absorption by a patient.

In one embodiment, the precursor glucagon-like peptide is the glucagon-like peptide analogue Semaglutide. In another embodiment, the precursor glucagon-like peptide is the glucagon-like peptide analogue Liraglutide. In yet another embodiment, the precursor glucagon-like peptide is the glucagon-like peptide analogue Tirzepatide.

In one embodiment, the glucagon-like peptide for the first incubation is provided at a concentration of 1.5 micromoles. In another embodiment the chelating agent is provided at a concentration range of between five and ten micromoles. The chelating agent may be ethylene-diaminetetraacetic acid (EDTA). In various other embodiments, the chelating agent may be dimercaprol, dimercaptosuccinic acid, and/or egtazic acid. Preferably, the zinc acetate is provided at a concentration range of between fifteen and one hundred micromoles.

In one embodiment, the glucagon-like peptide is incubated with the chelating agent for at least one hour. In another embodiment, the chelated glucagon-like peptide is incubated with the zinc acetate solution for at least eight hours. Preferably, the resulting zinc-glucagon-like peptide is packaged for oral administration in any conventional manner, including as pills or capsules.

Also disclosed is a method of treating diseases with a zinc-glucagon-like peptide composition for oral administration, the treatment method begins with preparing a zinc-glucagon-like peptide in any of the aforementioned ways, by first incubating a glucagon-like peptide (preferably an analogue to natural GLP-1) with a chelating agent, and then incubating the chelated product with zinc acetate. The resulting product is packaged for oral administration and orally administered to a patient, whereby the glucagon-like peptide is intestinally absorbed and enters the patient's blood stream. These and other features and advantages of the present invention will become appreciated as the same becomes better understood with reference to the specification, claims and drawings herein:

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates the results of an ELISA assay of mouse plasma from orally administered Liraglutide GLP-1 in response to control and blank results.

FIG. 2 illustrates ELISA assay results showing Liraglutide GLP-1 mouse sera and positive control versus a control mouse and negative control.

FIG. 3 illustrates ELISA assay results of the mouse plasma reflecting the presence of Liraglutide GLP-1 at one, one and one half, and eight hours relative to a control mouse.

FIG. 4 illustrates ELISA assay results of mouse plasma reflecting the presence of Liraglutide GLP-1 after a predetermined time.

FIG. 5 illustrates ELISA assay results of Liraglutide GLP-1 present in the pancreas of mice treated orally with Liraglutide GLP-1 versus control mice.

FIG. 6 illustrates the results of blood sugar levels from mice given orally administered Liraglutide GLP-1 versus control mice.

FIG. 7 illustrates the results of body weight changes in mice given orally administered Liraglutide GLP-1 versus control mice.

FIG. 8 illustrates the results of blood glucose levels in mice given orally administered Semaglutide GLP-1 versus control mice.

FIG. 9 illustrates the results of glucose tolerance in human subjects caused by orally administered Semaglutide GLP-1, compared with control human subjects.

FIG. 10 illustrates the results of an ELISA assay reflecting the existence of Semaglutide GLP-1 in the serum of mouse fed with Zn-Semaglutide.

FIG. 11 illustrates the results of an ELISA assay reflecting the presence of orally administered Semaglutide GLP-1 in the pancreas and brain of experimental mice relative to control mice.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so this disclosure will be thorough and complete, and will fully convey the scope of the invention to those skilled in the art.

It will be understood that when an element is referred to as being “on” another element, it can be directly on the other element or intervening elements may be present there between. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer, or section.

As used herein, the singular forms “a,” “an,” and “the,” are intended to include the plural forms as well, unless the context clearly indicates otherwise. It will be further understood that the terms “comprises” and/or “comprising,” “includes” and/or “including,” and “have” and/or “having,” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments of the present invention are described herein with reference to idealized embodiments of the present invention. As such, variations from the shapes of the illustrations as a result, for example, of manufacturing techniques and/or tolerances, are to be expected. Thus, embodiments of the present invention should not be construed as limited to the particular shapes of regions illustrated herein but are to include deviations in shapes that result, for example, from manufacturing.

Glucagon-like peptide-1 (GLP-1) is a physiological hormone having multiple actions on glucose. These actions are mediated by the GLP-1 receptors found on beta cells of the pancreas and on neurons of the brain. Specialized intestinal enteroendocrine cells known as L-cells, found in the lining of the intestines are the major source of the GLP-1 hormone. These L-cells may be found to a greater extent in the ileum and large intestine, and to a lesser extent in the duodenum and jejunum.

GLP-1 secretion encourages the release of insulin from the pancreas, and increases the volume of pancreatic beta cells that produce insulin. GLP-1 also causes a corresponding reduction in the release of glucagon, a peptide hormone produced in pancreatic alpha cells that raises glucose and fatty acid concentrations in the bloodstream. GLP-1 also increases satiety (the feeling of being full after eating) during and between meals by acting on appetite centers in the brain and by slowing the emptying of the stomach.

As a result of its secretion, GLP-1 has been shown to have the beneficial effects of lowing blood glucose and causing a reduction in body weight. However native GLP-1 has a very short plasma half-life and is rapidly metabolized. For this reason, GLP-1 analogs have been developed for use in treating diabetes and obesity, among other conditions. The three most recently approved GLP-1 analogs used to treat diabetes and obesity are Semaglutide (sold by Novo Nordisk under the trade name OZEMPIC®), Liraglutide (sold by Novo Nordisk under the trade names VICTOZA® and SAXENDA®), and Tirzepatide (sold by Eli Lilly under the trade names ZEPBOUND® and MOUNJARO®). These GLP-1 analogs act as GLP-1 receptor agonists that selectively bind to and activate GLP-1 receptors, the target for native GLP-1.

Each of these three GLP-1 analogs are peptides having an amino acid length of thirty one to thirty two amino acids. Due to the peptidic nature of these three medications, they must typically be administered via injection. Semaglutide, however, in combination with an absorption enhancer (for example, an intestinal permeation enhancer such as Salcaprozate sodium (SNAC)) has been used as an orally administered medicine. However, administered in this manner, Semaglutide has a bioavailability of only about 0.8 percent.

Disclosed herein is a technology called “Protein Surface Ions Modification” (PSIM). Using this technology, certain proteins have been successfully made to become orally absorbable with absorption rate at seventy to seventy five percent.

### Zinc Ion Modification of Liraglutide

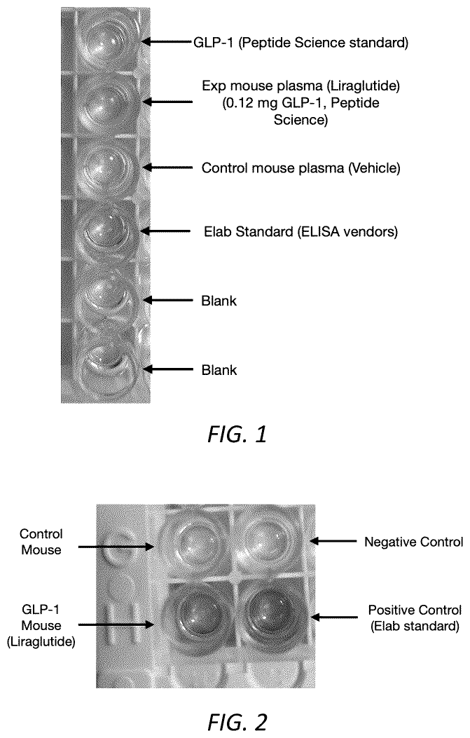

In an exemplary embodiment of the PSIM process, Liraglutide is dissolved in deionized water, and incubated with Ethylenediaminetetraacitic acid (EDTA) (or a similar chelating agent) for at least one hour, followed by incubation in a zinc salt solution, such as a zinc acetate solution or other zinc compound solution for at least eight hours. The concentrations of each reagent are as follows:
Liraglutide: 1.5 mM
EDTA: 5 to 10 mM
Zinc acetate: 15-100 mM
   Referring to FIGS. 1-7, an enzyme-linked immunosorbent assay (ELISA) was performed on the sera of mice orally administered with Liraglutide following the PSIM process.

FIG. 1 shows the results of an ELISA assay of the sera of mice orally administered with 0.12 mg Liraglutide. In this assay, a mouse (at two weeks old) was gavaged with Liraglutide in contrasts to a control mouse to which no Liraglutide was administered. After two hours, serum was collected, and an ELISA assay performed. The blue color shown in FIG. 1, which was found in the serum of the experimental mouse, but not in the serum of the control mouse suggests the existence of Liraglutide in the serum.

A quantitative ELISA assay to determine the absorption rate was also performed. The experimental mouse was sacrificed and whole body fluid collected one hour after feeding with Liraglutide. The intensity of the color was compared with that of a standard curve constructed by standard Liraglutide. The Liraglutide absorption rate was estimated to be seventy two percent. Referring to the wells shown in FIG. 1 from top to bottom, the top well is a 0.1 mg Liraglutide GLP-1 standard. The second well shows the experimental mouse plasma (serum of mouse orally given 0.12 mg Liraglutide). The third well shows the control mouse plasma. The fourth well shows the Elab (ELISA vendor) standard, and the bottom two wells are blanks.

FIG. 2 shows the results of an ELISA assay of the plasma of mice orally administered with Liraglutide as in FIG. 1. In FIG. 2, the top left well shows the ELISA assay result from sera of the control mouse, and the top right well represents the negative GLP-1 control. The bottom left well shows the ELISA assay result from sera of the mouse with orally administered Liraglutide, and the bottom right well shows the Elab positive control standard.

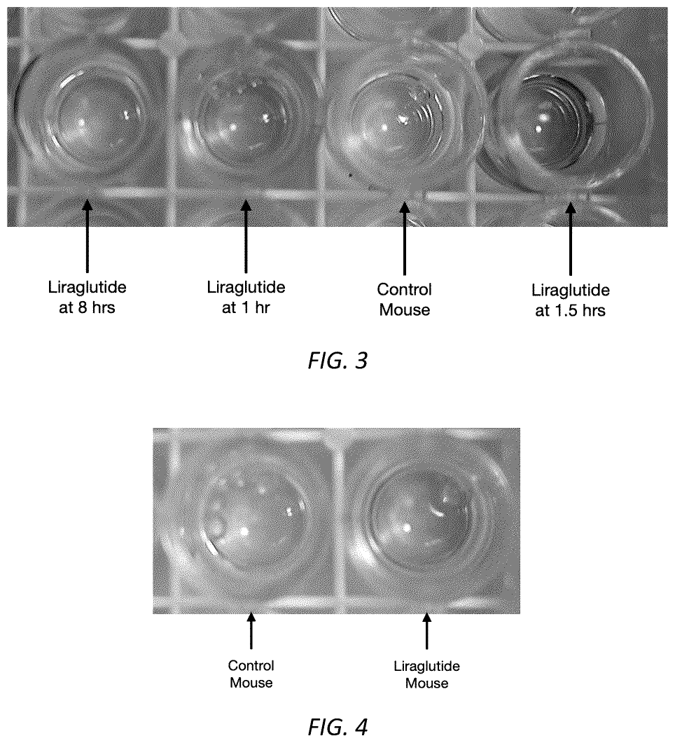

FIG. 3 shows the results of an ELISA assay of mouse plasma with orally administered Liraglutide measured over time. From left to right, the assay shows the prevalence of orally administered GLP-1 at 8 hours, at one hour, the absence of GLP-1 in the control mouse, and the presence of Liraglutide at 1.5 hours.

FIG. 4 shows the results of an ELISA assay of the sera of mice orally administered with Liraglutide as in FIG. 1. The well on the left shows the results of the control mouse, while the well on the right shows the results of the mouse treated with Liraglutide. In all instances in FIGS. 1-4 the mouse administered orally with Liraglutide showed an increased presence of GLP-1 even after an hour or more as reflected in the results of FIG. 3.

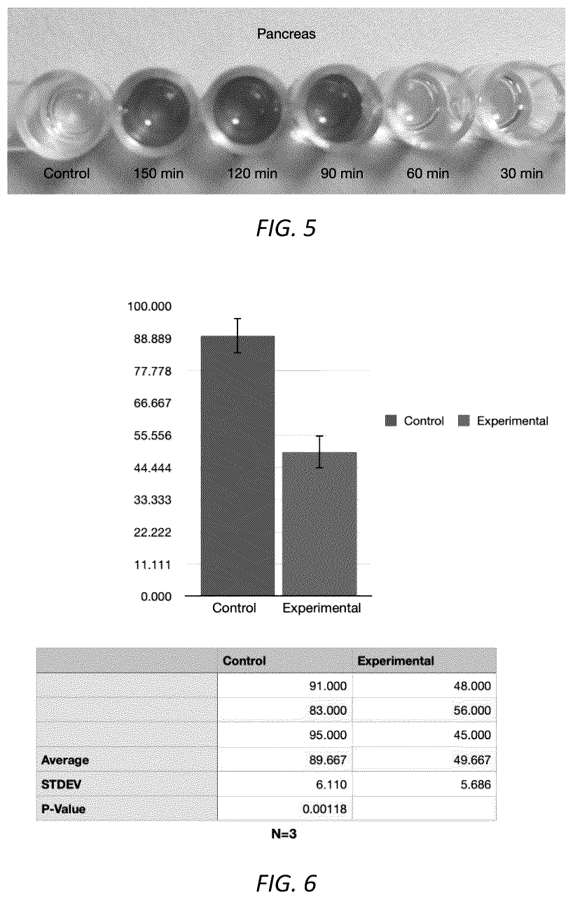

FIG. 5 shows the results of an ELISA assay of the pancreas of mice orally administered with Liraglutide. For this assay, mice were treated as in the assay of FIG. 1. A mouse at two weeks old was gavaged with Liraglutide, or not treated with Liraglutide to serve as a control mouse. Two hours later, the pancreas of the mouse was collected and homogenized, and a protein extract was centrifuged. Thereafter the ELISA essay was conducted. From left to right, the wells in the figure show a control mouse well, the results at 150 minutes, 120 minutes, 90 minutes, 60 minutes, and 30 minutes. The results indicate that Liraglutide appeared in the pancreas of the mouse ninety minutes after gavage with the Liraglutide formulation.

FIG. 6 shows the results of blood glucose levels from orally administered Liraglutide in mice. The orally administered Liraglutide was prepared by incubating Liraglutide at 1.7 mM in 9.6 mM EDTA for two hours, followed by incubation of the mixture with 15 mM zinc acetate for twelve hours to produce a Liraglutide for oral administration. The experimental mice were orally given 0.5 mg Liraglutide, and the control mice were administered a vehicle solution for 5 days. Thereafter, experimental and control mice were fasted for four hours followed by an orally given 50 mg glucose solution. Three and a half hours later, blood glucose was determined. The control mice were given a non-Liraglutide vehicle (N=3). The results show an elevated blood glucose in the control mice, reflecting an approximately 50% average difference in blood glucose (89.667:49.667) for control mice vs. mice with orally administered Liraglutide, with a standard deviation of 6.110 and 5.686, and a P-value of 0.00118.

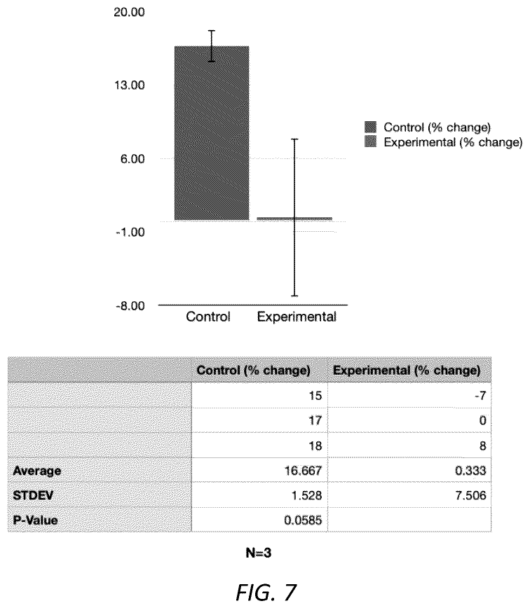

FIG. 7 shows the results of orally administered Liraglutide on the body weight of treated and control mice. The control group of mice was fed with a non-Liraglutide vehicle (N=3). The experimental group of mice were fed with the orally administered Liraglutide prepared in the manner discussed with regard to FIG. 6. Feeding was conducted over eight days. A dramatic difference in weight gain was shown, with the average percent change of the control mice being 16.667% and the average percent change of the experimental mice being 0.333%, with a standard deviation of 1.528 and 7.506, respectively, and a P-value of 0.0585.

### Zinc Ion Modification of Semaglutide

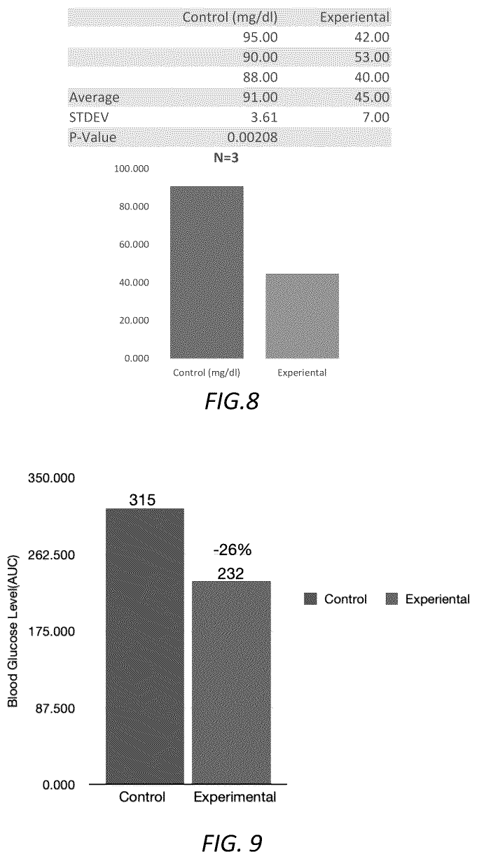

In another exemplary embodiment of the PSIM process, Semaglutide GLP-1 is dissolved in deionized water, and incubated with EDTA (or a similar chelating agent) for at least one hour, followed by incubation in a zinc acetate solution for at least eight hours. The concentrations of each reagent are as follows:
Semaglutide: 1.5 mM
EDTA: 5 to 10 mM
Zinc acetate: 15-100 mM
   Referring to FIGS. 8-12, an enzyme-linked immunosorbent assay (ELISA) was performed on the sera of mice orally administered with Semaglutide following the PSIM process.

FIG. 8 shows the results of blood glucose levels from orally administered Semaglutide GLP-1 in mice. The orally administered Semaglutide GLP-1 was prepared by incubating Semaglutide GLP-1 at 1.7 mM in 9.6 mM EDTA for two hours, followed by incubation of the mixture with 15 mM zinc acetate for twelve hours to produce a Semaglutide GLP-1 for oral administration. Experimental mice were orally given 0.5 mg Liraglutide, and control mice were given a vehicle solution for 5 days. Thereafter, experimental and control mice were fasted for four hours, followed by an orally given 50 mg glucose solution. Three and a half hours later blood glucose was determined. The control mice were given a non-Liraglutide vehicle (N=3). The results show an elevated blood glucose in the control mice, reflecting an approximately 50% average difference in blood glucose (91.00:45.00) for control mice vs. mice with orally administered Liraglutide, with a standard deviation of 3.61 and 7.00, and a P-value of 0.00208.

FIG. 9 shows the result of Semaglutide GLP-1 enhanced glucose tolerance in humans. A human subject was orally given 10 mg Semaglutide GLP-1 prepared as described in FIG. 8 at −2 minutes. A control human subject was not administered any Semaglutide GLP-1 At time zero, 15 g glucose was orally given. Blood glucose levels were monitored every fifteen minutes thereafter for ninety minutes. The area-under-curve (AUC), which represents the quantity of glucose in the blood was then determined. As shown in the graph, the control subject had an AUC of 315, while the subject with orally administered Semaglutide GLP-1 had an AUC of only 232, reflecting a twenty six percent AUC reduction.

The below Table shows the body weight changes between mice fed with Semaglutide GLP-1 (orally administered) and a control vehicle. A quantity of orally administered Semaglutide GLP-1 was prepared as discussed in FIG. 8, and orally given to mice (Exp.) for seven days, along with control mice (Control) to which no Semaglutide GLP-1 was orally administered. Body weight was measured at the beginning and on the seventh day. The results are reflected in the following table:

| | Body Weight | Body Weight | % Body |
|---|---|---|---|
| | (Day 1) | (Day 7) | Weight Change |
| Control 1 | 26 g | 30 g | +15% |
| Control 2 | 24 g | 28 g | +17% |
| Control 3 | 22 g | 26 g | +18% |
| Exp. 1 | 28 g | 26 g | −7% |
| Exp. 2 | 24 g | 26 g | +8% |
| Exp. 3 | 28 g | 28 g | +/−0% |

As shown from the data, the mice that were given orally administered Semaglutide GLP-1 showed a reduced weight gain or weight loss relative to the control mice.

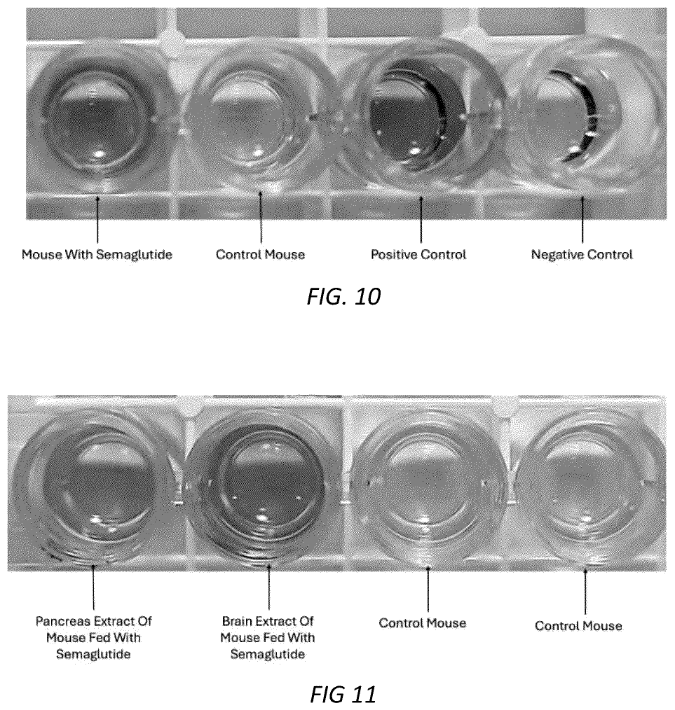

FIG. 10 shows the results of an ELISA assay of the sera of mice receiving orally administered Semaglutide GLP-1. A mouse, at two weeks old, was gavaged with Semaglutide GLP-1, a control mouse did not receive orally administered Semaglutide GLP-1. Two hours later, serum was collected, and an ELISA assay was performed. The blue color, which was found in the serum of experimental mouse but not in the serum of control mouse suggested that Semaglutide GLP-1 existed in the experimental mouse serum. A quantitative ELISA assay was performed to determine the absorption rate, and the mice were sacrificed and whole body fluid collected one hour after feeding with Semaglutide GLP-1. The results, as shown in the figure from left to right are the experimental mouse serum, the control mouse serum, a positive control, and a negative control. The intensity of the color was compared with that of a standard curve constructed by standard Semaglutide GLP-1. The absorption rate of orally administered Semaglutide GLP-1 was estimated to be approximately seventy percent.

FIG. 11 shows the results of an ELISA assay of the pancreas and brain extracts of mice receiving orally administered Semaglutide GLP-1. Experimental mice, at two weeks old, were gavaged with Semaglutide GLP-1 at 0.16 mg/mouse, and control mice received no orally administered Semaglutide GLP-1. Two hours later, the pancreas and brain were collected and homogenized, and protein extracts were subjected to an ELISA assay. In FIG. 11 from left to right, the wells show the brain extract of an experimental mouse, the pancreas extract of an experimental mouse, the brain extract of a control mouse, and the pancreas extract of a control mouse. The blue color, which was found in the extracts of pancreas and brain of experimental mouse but not in the those of control mouse suggested the presence of orally administered Semaglutide GLP-1 in the pancreas and brain of the experimental mice.

In one specific embodiment, Semaglutide at 1.7 micromoles concentration is incubated with EDTA at 9.6 micromoles concentration for two hours. The result is the incubated with zinc acetate at 15 micromoles concentration for twelve hours. In another specific embodiment, a human subject is orally given 10 mg Semaglutide prepared in any of the aforementioned manners for seven days.

### Zinc Ion Modification of Tirzepatide

In another exemplary embodiment of the PSIM process, Tirzepatide GLP-1 is dissolved in deionized water, and incubated with EDTA (or a similar chelating agent) for at least one hour, followed by incubation in a zinc acetate solution for at least eight hours. The concentrations of each reagent are as follows:
Tirzepatide: 1.5 mM
EDTA: 5 to 10 mM
Zinc acetate: 15-100 mM
   Weight loss and blood glucose tests were performed on the mice orally administered with Tirzepatide GLP-1 following the PSIM process, along with control mice to which no Tirzepatide GLP-1 was orally administered.

In one experiment, orally administered Tirzepatide GLP-1 caused weight loss in mice. A quantity of orally administered Tirzepatide GLP-1 was prepared as discussed above, and orally given to mice (Exp.) for five days, along with control mice (Control) to which no Tirzepatide GLP-1 was orally administered. Body weight was measured at the beginning and on the fifth day. The results are reflected in the following table:

| | Body Weight | Body Weight | % Body |
|---|---|---|---|
| | (Day 1) | (Day 7) | Weight Change |
| Control 1 | 20 g | 22 g | +10% |
| Control 2 | 22 g | 24 g | +9% |
| Control 3 | 26 g | 28 g | +7.7% |
| Exp. 1 | 26 g | 22 g | −18% |
| Exp. 2 | 24 g | 23 g | −4.1% |
| Exp. 3 | 28 g | 26 g | −7.1% |

As shown from the data, the mice that were given orally administered Tirzepatide GLP-1 showed a reduced weight gain or weight loss relative to the control mice.

Orally administered Tirzepatide GLP-1 was also shown to lower blood glucose levels in mice. The orally administered Tirzepatide GLP-1 was prepared in the manner discussed above. Thereafter 0.15 mg of orally administered Tirzepatide GLP-1 was fed to mice for five days. The mice were fasted for four hours, then 50 mg of a glucose solution was orally given. Three hours later, blood glucose levels were determined. In three control mice, who received no orally administered Tirzepatide GLP-1, the glucose levels in mg/dl were 93, 95, and 88, respectively. In three experimental mice that received orally administered Tirzepatide GLP-1, the glucose levels were 50, 52, and 47, respectively. The results reflect elevated blood glucose in the control mice vs. mice with orally administered Tirzepatide GLP-1, similar to orally administered Semaglutide GLP-1 and Liraglutide GLP-1 when subjected to the PSIM process.

When the zinc-GLP-1 is orally administered to a subject, it passes through the esophagus and enters the stomach. Non-zinc-bound GLP-1 analogues would not survive the acidic stomach conditions and would be dissolved or otherwise destroyed by hydrochloric acid and digestion enzymes present in the stomach. Zinc, however, is known to degrade hydrochloric acid by the chemical reaction: Zn+2HCl→ZnCl₂+H₂. Thus, the presence of the zinc results in the GLP-1 analogue being acid resistant, as the hydrochloric acid is neutralized by the zinc ion cloud rather than reacting with the GLP-1 polypeptide, thereby protecting it from digestion.

Additionally, acidic stomach conditions are necessary for peptic enzymes of the stomach to digest proteins. Thus, zinc ions neutralizing hydrochloric acid in the stomach help prevent the peptic enzymes from digesting the GLP-1 polypeptides. Thus, the orally administered zinc-GLP-1 analogue is resistant to both digestive enzymes as well as hydrochloric acid in the stomach, allowing the GLP-1 analogues to survive stomach digestion. The Zn-GLP-1 complex was then picked up and absorbed by zinc transporter proteins such as ZIP4 existing on the intestinal brush border membrane to be transported into the gastrointestinal (GI) tract. In the GI tract, the Zn-GLP-1 complex was then discharged into blood stream by another zinc transporter protein, ZnT1 existing on the basolateral membrane which faces the portal blood vessel. In the blood stream, the zinc ions may gradually release from the Zn-GLP-1 complex resulting the transport of intact GLP-1 into blood stream from the GI tract.

One preferred chelating agent for removing surface ions from the GLP-1 analogue is ethylenediaminetetraacetic acid (EDTA). Other chelating agents, however, including but not limited to dimercaprol, dimercaptosuccinic acid (DMSA), and egtazic acid (EGTA), may be utilized as chelating agents without departing from the concepts disclosed herein. While any concentration of chelating agent greater than 0 micromoles will result in removing some surface ions, it is preferred to use a concentration of between five and ten micromoles of the chelating agent.

While preferred ratios, concentrations, and compounds are discussed throughout the disclose of this method, other ratios, concentrations, and compounds may be utilized without departing from the general method disclosed herein.

1. A zinc-glucagon-like peptide composition for inducing an insulinotropic response in a patient through oral administration, the composition comprising:
a glucagon-like peptide comprising a polypeptidic chain of amino acids joined by peptide linkages ionically bound to zinc;
wherein the glucagon-like peptide is first incubated with a chelating agent comprising 5-10 mM EDTA, and thereafter incubated with a zinc salt solution;
wherein the zinc in the zinc salt solution is ionically bound to the chelated glucagon-like peptide, thereby creating the zinc-glucagon-like peptide for oral administration and intestinal absorption of the glucagon-like peptide.

2. The composition of claim 1 wherein the glucagon-like peptide is Semaglutide.

3. The composition of claim 1 wherein the glucagon-like peptide is Liraglutide.

4. The composition of claim 1 wherein the glucagon-like peptide is Tirzepatide.

5. The composition of claim 1 wherein the glucagon-like peptide is provided at a concentration of 1.5 mM.

6. (canceled)

7. The composition of claim 1 wherein the zinc in the zinc salt solution is provided at a concentration range of between fifteen and one hundred mM.

8. The composition of claim 1 wherein the glucagon-like peptide is incubated with the chelating agent for at least one hour.

9. The composition of claim 1 wherein the chelated glucagon-like peptide is incubated with the zinc salt solution for at least eight hours.

10. The composition of claim 1 wherein the zinc-glucagon-like peptide is packaged for oral administration.

11. A method of treating diseases with a zinc-glucagon-like peptide composition for oral administration, the treatment method comprising the steps of:
providing a glucagon-like peptide comprising a polypeptidic chain of amino acids joined by peptide linkages, the peptide dissolved in deionized water;
providing a chelating agent;
providing zinc in the form of a zinc salt solution;
incubating the glucagon-like peptide in the chelating agent for a predetermined time to form a chelated glucagon-like peptide;
incubating the chelated glucagon-like peptide with the zinc salt solution for a predetermined time; and
wherein the zinc is ionically bound to the chelated glucagon-like peptide, such that when the zinc-glucagon-like peptide is orally administered, the glucagon-like peptide is intestinally absorbed.

12. The method of claim 11 wherein the glucagon-like peptide is Semaglutide.

13. The method of claim 11 wherein the glucagon-like peptide is Liraglutide.

14. The method of claim 11 wherein the glucagon-like peptide is Tirzepatide.

15. The method of claim 11 wherein the glucagon-like peptide is provided at a concentration of 1.5 mM.

16. The method of claim 11 where wherein the chelating agent is provided at a concentration range of between five and ten mM.

17. The method of claim 11 wherein the zinc in the zinc salt solution is provided at a concentration range of between fifteen and one hundred mM.

18. The method of claim 11 wherein the glucagon-like peptide is incubated with the chelating agent for at least one hour.

19. The method of claim 11 wherein the chelated glucagon-like peptide is incubated with the zinc acetate solution for at least eight hours.

20. The method of claim 11 further comprising the step of packaging the zinc-glucagon-like peptide for oral administration.
